(19) United States
(12) Patent Application Publication
(10) Pub. No.: US 2025/0186769 A1
(43) Pub. Date: Jun. 12, 2025

(54) SELECTING SIZE OF TRANSDUCER FOR DELIVERING ALTERNATING ELECTRIC FIELDS

(71) Applicant: Novocure GmbH, Root D4 (CH)

(72) Inventors: Ariel NAVEH, Haifa (IL); Ari FRANK, Haifa (IL)

(73) Assignee: Novocure GmbH, Root D4 (CH)

(21) Appl. No.: 18/975,879

(22) Filed: Dec. 10, 2024

Related U.S. Application Data
(60) Provisional application No. 63/608,647, filed on Dec. 11, 2023.

(51) Int. Cl. A61N 1/36; G16H 10/60; G16H 20/40

(52) U.S. Cl. CPC A61N 1/36002; G16H 10/60; G16H 20/40

(57) ABSTRACT

A computer-implemented method for selecting transducers for delivering alternating electric fields to a subject, the method comprising: obtaining a three-dimensional model of at least a portion of the subject; determining a location of a region of interest (ROI) in the three-dimensional model; determining first and second potential surfaces of the three-dimensional model for placement of first and second transducers on the subject based on the ROI; and selecting a first pair of transducers from a plurality of transducers to deliver alternating electric fields to the ROI at selected locations on the three-dimensional model based on the location of the ROI in the three-dimensional model and the potential surfaces of the three-dimensional model, wherein the plurality of transducers comprises a small transducer and a large transducer having an area larger than an area of the small transducer.

### CROSS-REFERENCE TO RELATED APPLICATIONS

This application claims priority to U.S. Provisional Patent Application No. 63/608,647, filed Dec. 11, 2023, which is incorporated herein by reference in its entirety.

### BACKGROUND

Tumor treating fields (TTFields) are low intensity alternating electric fields within the intermediate frequency range (for example, 50 kHz to 1 MHZ), which may be used to treat tumors as described in U.S. Pat. No. 7,565,205. TTFields are induced non-invasively into a region of interest by electrode assemblies (also known as electrode arrays, transducer arrays, or simply “transducers”) placed on the patient's body and applying alternating current (AC) voltages between the transducers. Conventionally, one or more pairs of transducers (e.g., a first pair of transducers and a second pair of transducers) are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 illustrates a flowchart depicting an example computer-implemented method for selecting transducers for delivering an alternating electric fields to a subject.

FIG. 2 depicts examples of transducers for delivering alternating electric fields to a subject.

FIGS. 3A, 3B, and 3C depict examples of selecting transducers for delivering alternating electric fields to a subject.

FIGS. 4A and 4B depict examples of selecting transducers for delivering alternating electric fields to a subject.

FIG. 5 illustrates a flowchart depicting an example method for applying TTFields to a subject.

FIG. 6 illustrates a flowchart depicting an example computer-implemented method for selecting transducers for delivering alternating electric fields to a subject.

FIG. 7 illustrates an example apparatus to apply alternating electric fields to a subject's body.

FIGS. 8A and 8B illustrate schematic views of exemplary design of a transducer for applying alternating electric fields.

FIG. 9 illustrates an example computer apparatus.

### DESCRIPTION OF EMBODIMENTS

This application describes exemplary techniques to computationally determine where to place transducers on a subject.

In general, one or more pairs of transducers are positioned on the subject's body and used to apply alternating electric fields to the subject's body. In some embodiments, at least two pairs of transducers are used. Each transducer may have one or more electrode elements on a substrate and may be capable of administering TTFields to the subject's body. Determining where to place the transducers on the subject involves determining one or more transducer layouts, where each transducer layout specifies where to place each transducer on the subject. For a particular transducer layout, existing techniques use computationally intensive algorithms to determine current flow in a three-dimensional model of a subject based on conductivities of tissue types assigned to voxels in the three-dimensional model of the subject and calculate predicted energy delivered to a region of interest (ROI) (e.g., an area with a tumor) of the subject. These computationally intensive algorithms run electromagnetic simulations for each transducer layout considered for the subject.

The inventors discovered how to select transducer layouts based on high-level criteria without needing to run such computationally intensive algorithms for each transducer layout. With the inventive techniques, transducer layouts may be determined based on a proximity of a region of interest (ROI) (e.g., based on a tumor of the subject) to the exterior surface (e.g., skin) of the subject and/or the size of the ROI. Further, transducers of different sizes may be considered for the transducer layouts, where a smaller transducer may provide better and/or more precise current delivery to the ROI and where a larger transducer may help to better target the treatment area.

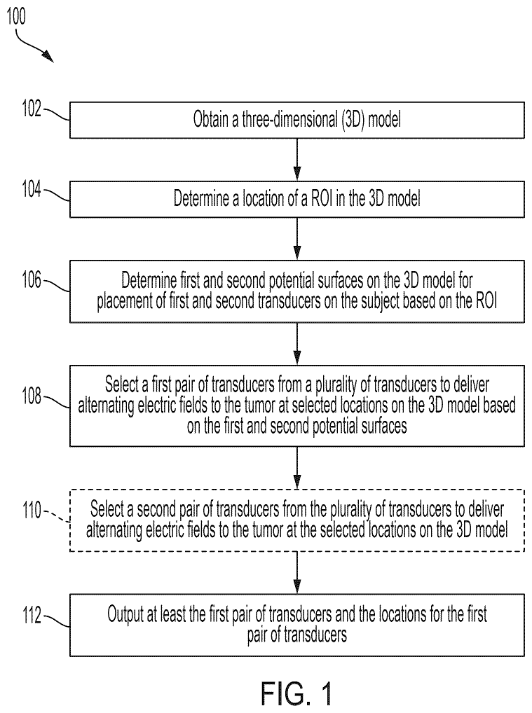

FIG. 1 is a flowchart depicting an example computer-implemented method for selecting transducers for delivering alternating electric fields to a subject. In method 100, the transducers and the transducer layouts are determined via computer-implemented geometrical calculations of the subject. Certain steps of the method 100 are described as computer-implemented steps. The computer may be, for example, any device comprising one or more processors and memory accessible by the one or more processors, the memory storing instructions that when executed by the one or more processors cause the computer to perform the relevant steps of the method 100. The method 100 may be implemented by any suitable system or apparatus, such as the apparatus of FIG. 9. While an order of operations is indicated in FIG. 1 for illustrative purposes, the timing and ordering of such operations may vary where appropriate without negating the purpose and advantages of the examples set forth in detail herein.

With reference to FIG. 1, at step 102, the method 100 may include obtaining a three-dimensional (3D) model of at least a portion of the subject. The 3D model may have voxels, surface meshes, or volumetric meshes. As one example, each voxel, surface mesh, or volumetric mesh of the model may be assigned a type of tissue (e.g., bone, organs, fluid, skin, or tumor). The 3D model may be obtained from computer memory (or computer storage) locally or over a network. The 3D model may be generated based on one or more images of a region of the subject. The 3D model may also be a generic model representing basic and/or general features of the subject. In some embodiments, the one or more images are medical images. The medical image may include, for example, at least one of a magnetic resonance imaging (MRI) image, a computerized tomography (CT) image, an X-ray image, an ultrasound image, a nuclear medicine image, a positron-emission tomography (PET) image, arthrogram images, myelogram images, or any image of the subject's body providing an internal view of the subject. Each medical image may include an outer shape of a portion of the subject and a region corresponding to a region of interest (ROI) (e.g., a tumor, a region larger than and encompassing the tumor, or a region around the tumor but smaller than the tumor) within the subject. In some embodiments, the ROI may be a location in the subject to direct the administration of alternating electric fields (e.g., TTFields). As an example, the medical image may be a 3D MRI image. As an example, the 3D model may represent a head of the subject. As another example, the 3D model may represent a torso of the subject. Other body parts of the subject may be represented in the model of the subject in other embodiments.

At step 104, the method 100 may include determining a location of a ROI in the 3D model. In some embodiments, the ROI may be the same size and shape as a tumor in the subject, may be larger than and encompassing the tumor, or may encompass a portion of the tumor. As an example, the location of the ROI may be determined based on voxels or mesh in the obtained 3D model. The location of the ROI may be determined automatically and/or based on user input.

At step 106, the method 100 may include determining first and second potential surfaces on the 3D model for placement of first and second transducers on the subject based on the ROI. In some embodiments, this determination may include determining the first potential surface on the 3D model based on a location of the ROI, and determining the second potential surface on the 3D model based on the first potential surface and the location of the ROI. In some embodiments, the first potential surface and the second potential surface may include one or more points or areas on the surface of the 3D model of the subject. In some embodiments, the potential surfaces may not include one or more avoidance areas of the subject (e.g., a nipple, a surgical scar, an eye, an ear, a mouth, a nose, irritated skin, a sensitive area, etc.).

In some embodiments, the potential surfaces of the 3D model may be determined based on computer geometric calculations comparing voxels on the surface of the 3D model of the subject to voxels of the ROI in the 3D model of the subject. As an example, the first and second potential surfaces of the 3D model may be determined based on distances between surfaces of the 3D model and the ROI. As an example, the first and second potential surfaces of the 3D model may be determined based on a centroid of the ROI, an exterior surface of the ROI, or a shape enclosing the ROI (e.g., a convex shape or a concave shape). In some embodiments, the potential surfaces may be determined among a plurality of potential surfaces based on criteria including providing highest dosage in the ROI (e.g. highest field intensity), providing maximized patient comfort, avoiding an avoidance area, etc.

In some embodiments, determining the first and second potential surfaces of the 3D model may include determining a pair of first and second points on the 3D model, where the first point is closer to the ROI than the second point, where the first potential surface includes the first point, and where the second potential surface includes the second point. In some embodiments, determining the pair of first and second points on the 3D model may exclude points on the surface of the 3D model that correspond to an avoidance area of the subject. As an example, the first point may be closer to the ROI than the second point based on a percentage of the distance between the second point and the ROI. For example, if the distance between first point and the ROI needs to be 20% or less than the distance between the second point and the ROI, and if the distance between the second point and the ROI is 15 cm, then the first point needs to be within 3 cm of the ROI. In some embodiments, this percentage may be based on user input, and the percentage may be, for example, 10%, 20%, 30%, 40%, or 50%, or any percentage therebetween.

In some embodiments, determining the first potential surface of the 3D model may include determining a first point on the surface of the 3D model of the subject with respect to a portion of ROI, where the first potential surface includes the first point. In some embodiments, determining the first point on the surface of the 3D model may exclude points on the surface of the 3D model that correspond to an avoidance area of the subject. As an example, the portion of the ROI used to the determine the first point of the 3D model may be a centroid of the ROI, an exterior surface of the ROI, or a shape enclosing the ROI. As one example, the first point may be a point having a distance to the centroid of the ROI that is smaller than any distances between any other points on the surface of the 3D model and the centroid of the ROI. As another example, an average distance between points on the first potential surface and the centroid of the ROI is smaller (or at least, for example, 5%, 10%, 15%, or 20% smaller, or any percentage therebetween) than an average distance between points on any other surfaces and the centroid of the ROI.

In some embodiments, once the first potential surface is determined, the second potential surface may be determined based on the first potential surface and the ROI. As an example, once the first potential surface is determined, the second potential surface may be determined as an area on the 3D model that is opposite to the first potential surface, where the ROI is located between the first and second potential surfaces. As an example, once the first potential surface is determined, the second potential surface may be determined as an area on the 3D that may form a line or an energy channel through the ROI and with the first potential surface. As an example, the first potential surface may be on the front torso of the subject, and the second potential surface may be on the back torso of the subject. As an example, the first potential surface may be on the front left torso of the subject, and the second potential surface may be below the right armpit on the side of the torso of the subject.

At step 108, the method 100 may include selecting a first pair of transducers from a plurality of transducers to deliver alternating electric fields to the ROI at selected locations on the 3D model based on the first and second potential surfaces. In some embodiments, the selected locations may be the first and second potential surfaces or locations with areas that include or a portion of the first and second potential surfaces. In some embodiments, the plurality of transducers in step 108 may be stored in memory, such as, for example, memory local to the computer or memory accessible over a network. In some embodiments, the plurality of transducers in step 108 may include transducers of different sizes. The size of a transducer may be an area of the transducer. As an example, when viewed from a direction perpendicular to a surface of a transducer, an area of the transducer may be an area of the transducer to emanate an electric field when the transducer is adjacent to a surface of the 3D model. As an example, when viewed from a direction perpendicular to a surface of a transducer, an area of the transducer may be a summation of areas of all electrodes of the transducer. As an example, when viewed from a direction perpendicular to a surface of the transducer, an area of the transducer may be an area of a smallest convex shape enclosing all electrodes of the transducer. In some embodiments, each transducer in the plurality of transducers may be modeled to provide a same amount of current to the subject.

As an example, the plurality of transducers may include at least a small transducer and a large transducer. As an example, the area of the small transducer may be about 50% to about 70% of the area of the large transducer, or the area of the small transducer may be at least 50% and at most 70% of the area of the large transducer. As an example, when viewed from a direction perpendicular to a surface of the transducer, an area of at least one electrode element of a small transducer may range from approximately 150 cm² to approximately 265 cm², and an area of at least one electrode element of a large transducer may range from approximately 300 cm² to approximately 525 cm². As an example, when viewed from a direction perpendicular to a surface of the transducer, an area of at least one electrode element of a small transducer may be at least 150 cm² and at most 265 cm², and an area of at least one electrode element of a large transducer may be at least 300 cm² and at most 525 cm². In some embodiments, the plurality of transducers may further include at least one mid-sized transducer having an area larger than the area of the small transducer and smaller than the area of the large transducer. The plurality of transducers may also include transducers and/or electrode elements of different shapes.

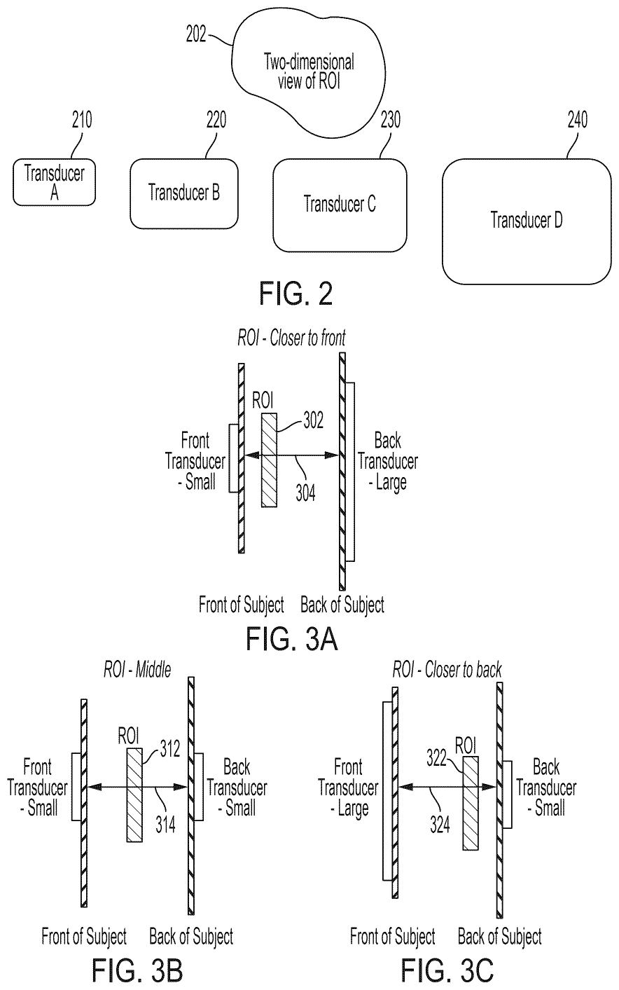

FIG. 2 illustrates a plurality of exemplary transducers of different sizes but having similar shapes for delivering alternating electric fields to a subject. The plurality of transducers may include at least one small transducer (e.g., transducer A 210), one mid-sized transducer (e.g., transducer B 220 and/or transducer C 230), and one large transducer (e.g., transducer D 240). Transducer B 220 and/or transducer C 230 may have an area larger than an area of transducer A 210 and smaller than an area of transducer D 240.

In some embodiments, the selected first pair of transducers may have a first transducer and a second transducer. The first transducer may be located at a first location on the surface of the 3D model, and the second transducer may be located at a second location on the surface of the 3D model. The first location of the first transducer may be selected to be located based on the first potential surface of the 3D model, and the second location of the second transducer may be located based on the second potential surface of the 3D model.

In some embodiments, selecting the first pair of transducers from the plurality of transducers may be based on the location of the ROI in the 3D model and the potential surfaces of the 3D model and/or based on a size of the ROI in relation to the closest surface of the 3D model.

As an example, selecting the first pair of transducers from the plurality of transducers may be based on an area of the ROI. For example, the area of the ROI may be calculated as an area of the ROI when the ROI is viewed from a direction approximately perpendicular to the first potential surface of the 3D model. As such, the 3D ROI may be calculated as a two-dimensional (2D) surface from a direction approximately perpendicular to the first potential surface of the 3D model. The first transducer of the first pair of transducers may be selected as the transducer from the plurality of transducers having an area closest to the area of the ROI. As such, upon determining the area of the ROI, a transducer is selected from the plurality of transducers having an area closest to the area of the ROI as the first transducer of the first pair of transducers, and the first location of the first transducer is selected based on the first potential surface of the 3D model.

Referring back to FIG. 2, FIG. 2 also shows a two-dimensional view of a ROI 202. In the example illustrated in FIG. 2, transducer C 230 may have an area closest to an area of the ROI 202 and may be selected as a first transducer of a first pair of transducers.

In some embodiments of selecting a first pair of transducers in step 108 of FIG. 1, a small transducer from the plurality of transducers may be selected as the first transducer of the first pair of transducers, and either the small transducer or a large transducer may be selected as the second transducer of the first pair of transducers. In some embodiments, the small transducer may be the smallest transducer in the plurality of transducers or may be a medium-sized transducer in the plurality of transducers but smaller than the large transducer. In some embodiments, the large transducer may be the largest transducer in the plurality of transducers or may be a medium-sized transducer in the plurality of transducers but larger than the small transducer.

In some embodiments, the second transducer of the first pair of transducers may be selected from the plurality of transducers based on a proximity of the ROI to the potential surfaces of the 3D model. As an example (see, e.g., FIG. 3A or FIG. 3C), the second transducer may be selected to be the large transducer if the ROI is closer to the first potential surface of the 3D model than to midway between the first location and the second location. As an example (see, e.g., FIG. 3B), the second transducer may be selected to be the small transducer if the ROI is closer to midway between the first location and the second location on the 3D model. As an example (see, e.g., FIG. 3B), if the ROI is closer to midway between the first location and the second location on the 3D model, the first transducer and the second transducer may be the same transducer from the plurality of transducers. As an example (see, e.g., FIG. 3B), if the ROI is closer to midway between the first location and the second location on the 3D model, both the first transducer and the second transducer may be the small transducer or a medium-sized transducer from the plurality of transducers.

In some embodiments, if a small transducer from the plurality of transducers is selected as the first transducer of the first pair of transducers, the second transducer of the first pair of transducers may be selected based on a line passing through a center, about the center, a centroid, or about the centroid of the ROI and between a first side of the subject (for the first location of the first transducer) and a second side of the subject (for the second location of the second transducer). As an example, the second transducer may be selected to be the small transducer if the ROI is located at about the center of the line (see, e.g., FIG. 3B), or may be selected to be the large transducer otherwise (see, e.g., FIG. 3A or FIG. 3C). As an example, the second transducer may be selected to be the large transducer if the ROI is located away from the center of the line (see, e.g., FIG. 3A or FIG. 3C), or may be selected to be the small transducer otherwise (see, e.g., FIG. 3B).

As an example, the second transducer may be selected to be the small transducer if at least about 50% of the ROI is located within about 10% of the center of the line, or the large transducer otherwise. As an example, the second transducer may be selected to be the small transducer if at least about 50%, 60%, 70%, or 80%, or any percentage therebetween, of the ROI is located within about 10%, 15%, 20%, or 25%, or any percentage therebetween, of the center of the line, or the large transducer otherwise.

As an example, the second transducer may be selected to be the large transducer if at least about 50% the ROI is located more than about 10% from the center of the line, or the small transducer otherwise. As an example, the second transducer may be selected to be the large transducer if at least about 50%, 60%, 70%, or 80%, or any percentage therebetween, of the ROI is located more than about 10%, 15%, 20%, or 25% or any percentage therebetween, from the center of the line, or the large transducer otherwise.

FIGS. 3A, 3B, and 3C depict examples of selecting transducers for delivering alternating electric fields to a subject for these example embodiments. For these examples, the plurality of transducers may have a small transducer and a large transducer, which has a larger area than an area of the small transducer. FIG. 3A illustrates an example where a ROI 302 is closer to the front of a 3D model of a subject, so that a front surface is determined based on a first potential surface of the 3D model. A line 304 may be calculated as passing through a center, about the center, a centroid, or about the centroid of the ROI 302 and between the front subject and the back of the subject. In some embodiments, the line 304 may be calculated as being perpendicular or substantially perpendicular to a surface of the ROI 302. The ROI 302 may be determined to be closer to the front of the 3D model based on the location of the ROI 302 with respect to the line 304. Here, at least about 50% the ROI 302 is located more than about 10% from the center of the line 304. For this example, the small transducer may have an area closest to the area of the ROI 302, and as such, the small transducer is selected as the first transducer of the first pair of transducers, where the first transducer is located based on the first potential surface. The large transducer is then selected as the second transducer of the first pair of transducers, where the second transducer is located based on the second potential surface (i.e., the back of the subject), which is a side opposite to the first potential surface in this example.

FIG. 3B illustrates an example where a ROI 312 is located near midway between the front and the back of a 3D model of a subject. In this example, the ROI 312 is located at about a center of a line 314 passing through the ROI 312 and between the front of the subject and the back of the subject. Here, at least about 50% ROI 312 is located within about 10% from the center of the line 314. For this example, the small transducer is selected as the first transducer of the first pair of transducers located at the front of the subject, and the small transducer is also selected as the second transducer of the first pair of transducers located at the back of the subject. As an example, when a ROI 312 is located near midway between the front and the back of a 3D model of a subject, a medium transducer or a large transducer may be selected as both the first transducer and the second transducer.

FIG. 3C illustrates an example similar to FIG. 3A, where a ROI 322 is closer to the back of a 3D model of a subject, so that a back surface is determined as a first potential surface of the 3D model. In this example, the ROI 322 is located away from a center of a line 324 passing through the ROI 322 and between the front of the subject and the back of the subject. Here, at least about 50% the ROI 322 is located more than about 10% from the center of the line 324. For this example, the small transducer may have an area closest to the area of the ROI 322, and as such, the small transducer is selected as a first transducer of a first pair of transducers, where the first transducer is located based on the first potential surface (i.e., the back of the subject). The large transducer is selected as the second transducer of the first pair of transducers, where the second transducer is located on a second potential surface (i.e., the front of the subject), which is a side opposite to the first potential surface.

In some embodiments, for step 108 in FIG. 1, selecting the first pair of transducers from the plurality of transducers may be based on a projection of the ROI onto the first potential surface of the 3D model. When viewed from a direction perpendicular or approximately perpendicular to the first potential surface of the 3D model, an area of the ROI may be calculated from a projection of the ROI onto the first potential surface of the 3D model, and the transducer selected as the first transducer of the first pair of transducers may have an area closest to the area of the projection of the ROI. The projection of the ROI onto the first potential surface may be based on at least one line intersecting a perimeter of the second transducer and a perimeter of the ROI. Specifically, when viewed from a direction perpendicular or approximately perpendicular to the first potential surface of the 3D model, the projection of the ROI onto the first potential surface may be calculated, and the area of the ROI may be calculated as the area inside the projection. Upon determining the projection of the ROI onto the first potential surface, the area of the ROI may be calculated, and a transducer may be selected from the plurality of transducers having a size closest to a size of the projection of the ROI onto the first potential surface (e.g., an area closest to an area determined from the projection). This selected transducer may be the first transducer of the first pair of transducers, and a first location of the first transducer may be selected to be on the first potential surface of the 3D model based on a location of the projection of the ROI onto the first potential surface.

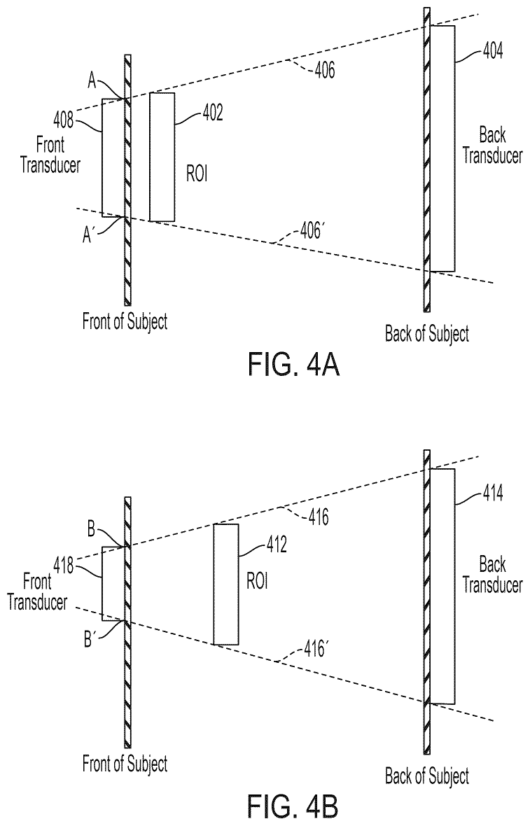

FIGS. 4A and 4B depict examples of selecting transducers for delivering alternating electric fields to a subject. FIG. 4A illustrates an example of selecting a first transducer of a first pair of transducers based on a projection of a ROI onto the first potential surface. As illustrated in FIG. 4A, the ROI 402 is located closer to the front of the subject, and so, the front surface of the subject is determined to be the first potential surface. A second transducer 404 (i.e., the back transducer) is selected to be a large transducer at the back of the subject. A projection of the ROI is based on two projection lines 406 and 406′ intersecting a perimeter of the ROI 402 and a perimeter of the second transducer 404 at points A and A′, respectively, on the front surface of the subject. The projection of the ROI is determined based on a distance between points A and A′. For illustration purposes, the projection of the ROI onto the first potential surface is shown being determined from points A and A′. However, the projection of the ROI may be determined based on more than two points on the first potential surface from more than two lines intersecting the perimeter of the ROI 402 and the perimeter of the second transducer 404. An area of the ROI may be determined from the projection. A transducer 408 may be selected as the first transducer, and the first location of the first transducer may be determined at the projection position. In some embodiments, the transducer 408 may have a size that is closest to the size of the projection. In some embodiments, the transducer 408 may have a size that is smaller than, equal to, or larger than the size of the projection.

Similar to FIG. 4A, FIG. 4B illustrates an example of selecting a first transducer of a first pair of transducers based on a projection of a ROI onto the first potential surface. In FIG. 4B, however, the ROI 412 is farther from the front of the subject than the ROI 402 in FIG. 4A. A second transducer 414 (i.e., the back transducer) is selected to be a large transducer at the back of the subject. A projection of the ROI is based on two projection lines 416 and 416′ intersecting a perimeter of the ROI 412 and a perimeter of the second transducer 414 at points B and B′, respectively, on the front surface of the subject. An area of the ROI may be determined from the projection. A transducer 418 may be selected as the first transducer, and the first location of the first transducer may be determined at the projection position. In some embodiments, the transducer 418 may have a size that is closest to the size of the projection. In some embodiments, the transducer 418 may have a size that is smaller than, equal to, or larger than the size of the projection. As can be seen in comparing FIG. 4A and FIG. 4B, transducer 408 is larger than transducer 418. So, for this example, as the location of a ROI is further away from the front of the subject (and closer to midway between the front and the back of the subject), the projection of the ROI may become smaller, and thus, a smaller transducer may be selected as the first transducer.

While FIGS. 4A and 4B illustrate an example with the back transducer being the larger transducer, another example may have the front transducer being the larger transducer with the projection lines converging towards the back of the subject.

Referring back to FIG. 1, at step 110, the method 100 may include selecting a second pair of transducers from the plurality of transducers to deliver alternating electric fields to the ROI at the selected locations on the 3D model. In some embodiments, the second pair of transducers may be selected using a technique similar to how the first pair of transducers was selected in step 108. In some embodiments, the second pair of transducers may be selected using a known technique. In some embodiments, step 110 may be optional since only one pair of transducers is used in the TTFields treatment planning. As such, step 110 is shown in a dashed line to indicate the optional use of step 110 in the method 100.

At step 112, the method 100 may include outputting a representation of the first pair of transducers and locations for the first pair of transducers on the subject. If determined in step 110, a representation of the second pair of transducers and locations for the second pair of transducers on the subject may also be outputted. As an example, a display is used to show a representation of the first transducer and/or the first location on the subject. As an example, a display is used to show a representation of the second transducer and/or the second location on the subject. As an example, a display is used to show a representation of the first pair of transducers and/or the first and second locations on the subject. As an example, a display is used to show a representation of the first pair of transducers on the subject and the second pair of transducers on the subject. As an example, a display is used to show a representation of locations for the first pair of transducers on the subject and locations for the second pair of transducers on the subject. As an example, the output of the representation for step 112 may be in form of a document.

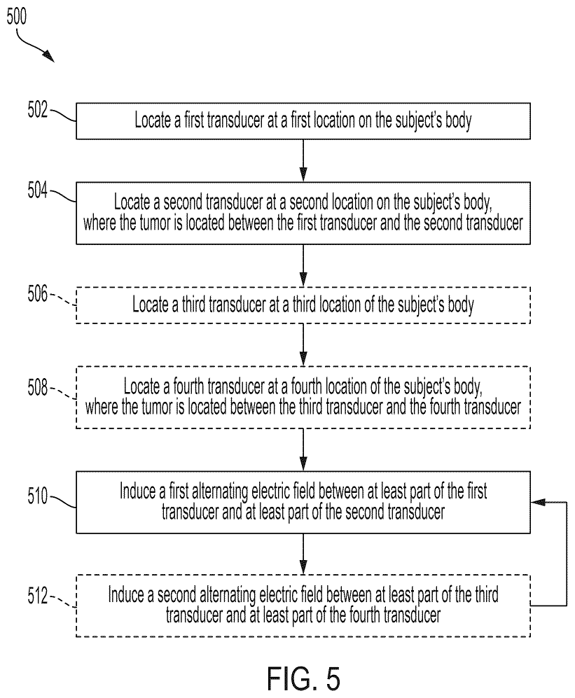

FIG. 5 illustrates a flowchart depicting an example method 500 for applying TTFields to a subject. Using the method of FIG. 1, the first and second pairs of transducers and their respective locations on the subject may be selected, and using the method of FIG. 5, TTFields may be administered to the subject. Certain steps of the method 500 are described as computer-implemented steps. The computer may be, for example, any device comprising one or more processors and memory accessible by the one or more processors, the memory storing instructions that when executed by the one or more processors cause the computer to perform the relevant steps of the method 500. The method 500 may be implemented by any suitable system or apparatus, such as the apparatus of FIG. 9. While an order of operations is indicated in FIG. 5 for illustrative purposes, the timing and ordering of such operations may vary where appropriate without negating the purpose and advantages of the examples set forth in detail herein.

With reference to FIG. 5, at step 502, the method 500 may include locating a first transducer at a first location of the subject's body. The first transducer and the first location in step 502 may be the first transducer and the first location selected in method 100. The first transducer may be a smaller transducer. In some embodiments, the first transducer may have at least one electrode element adapted to be coupled to a voltage generator.

At step 504, the method 500 may include locating a second transducer at a second location of the subject's body to pair with the first transducer at the first location of step 502 for administering TTFields to the subject. The second transducer and the second location in step 504 may be the second transducer and the second location selected in method 100. As such, the first transducer at the first location and the second transducer at the second location together may form a first pair of transducers. The second transducer of step 504 may be a larger transducer and/or may be larger than the first transducer of step 502. The second transducer of step 504 may be a small transducer similar to the first transducer of step 502. As an example, a ROI may be located between the first transducer and the second transducer. In some embodiments, the second transducer may have at least one electrode element adapted to be coupled to the voltage generator.

At step 506, the method 500 may optionally include locating a third transducer at a third location of the subject's body. At step 508, the method 500 may optionally include locating a fourth transducer at a fourth location of the subject's body to pair with the third transducer at the third location of step 506 for administering TTFields to the subject. As such, the third transducer at the third location and the fourth transducer at the fourth location together may compose a second pair of transducers. In some embodiments, the ROI of the subject may be located between the third transducer and the fourth transducer. In some embodiments, partial of the third or fourth transducer may be overlapped with the first or second location, for example, adhesive areas at edges the transducers. In some embodiments, the location of the third or fourth transducer may not be overlapped with the first or second location. As an example, all transducers (e.g., first transducer, second transducer, third transducer, and fourth transducer) may target the same ROI for additional or synergistic therapeutic effects of the alternating electric fields.

At step 510, the method 500 may include inducing a first alternating electric field between at least part of the first transducer and at least part of the second transducer by applying an AC voltage between this first pair of transducers. At step 512, the method 500 may optionally include inducing a second alternating electric field between at least part of the third transducer and at least part of the fourth transducer by applying an AC voltage between this first pair of transducers. Flow cycles between steps 510 and 512 to generate the alternating electric field at a particular interval for a particular period of time depending on a determined TTFields dosage.

In some embodiments, if only a pair of transducers is used for the alternating electric fields treatment, steps 506, 508, and 512 may not be performed.

As an example, an alternating electric field (e.g., TTFields) may be applied to the ROI, tumors, cells, or the area of a subject. In some embodiments, the alternating electric field may be applied with predetermined parameters. As an example, the alternating electric field may include a frequency within a frequency range from about 50 kHz to about 1 MHz, or include a frequency of at least 50 kHz and at most 1 MHz. As an example, the alternating electric field may include a frequency within a frequency range from about 50 kHz to about 10 MHz, or include a frequency of at least 50 kHz and at most 10 MHz. As an example, the frequency of the alternating electric field may be: between about 50 kHz and about 1000 kHz; or between about 100 kHz and about 300 kHz; or at least 50 kHz and at most 1000 kHz; or at least 100 kHz and at most 300 kHz. As an example, the frequency of the alternating electric field may be about 100 kHz, about 150 kHz, about 200 kHz, about 250 kHz, or about 300 kHz, or any frequency therebetween. As an example, the frequency of the alternating electric field may be 100 kHz, 150 kHz, 200 kHz, 250 kHz, or 300 kHz, or any frequency therebetween.

As an example, the alternating electric fields (e.g., TTFields) may be given an intensity within a range up to about 10 V/cm or up to 10 V/cm. As an example, the intensity of the alternating electric fields may be between about 1 V/cm and about 4 V/cm, or may be at least 1 V/cm and at most 4 V/cm. As an example, the intensity of the alternating electric fields may be between about 1 V/cm and about 4 V/cm, or may be at least 1 V/cm and at most 4 V/cm. Other possible exemplary parameters for the alternating electric field may include active time, dimming time, and duty cycle (all of which may be measured in, for example, ms units), among other parameters. The parameters may be modified based on the conditions of the subject (e.g., the sizes of the tumor, type of tumor, age, or sex of the subject) or the purposes of the treatment. As an example, the intensity of the alternating electric field may be between about 1 V/cm and about 4 V/cm, and the frequency of the alternating electric field may be between about 150 kHz and about 250 kHz for treating tumor/cancer cells. In some embodiments, the alternating electric field may be applied using two pairs of transducer arrays placed on the subject and directed on the tumor of the subject.

Various combinations of pairs of transducers, as discussed herein, or similar pairs of transducers may be used together. Various locations of transducers, such as those discussed herein or in other locations, may be used. A transducer may be used in a single pair of transducers or in two or more pairs of transducers. A transducer may be partitioned to be used in a single pair of transducers or in two or more pairs of transducers. The transducers, the transducer locations, the pairs of transducers, and the two or more pairs of transducers discussed herein are not exhaustive.

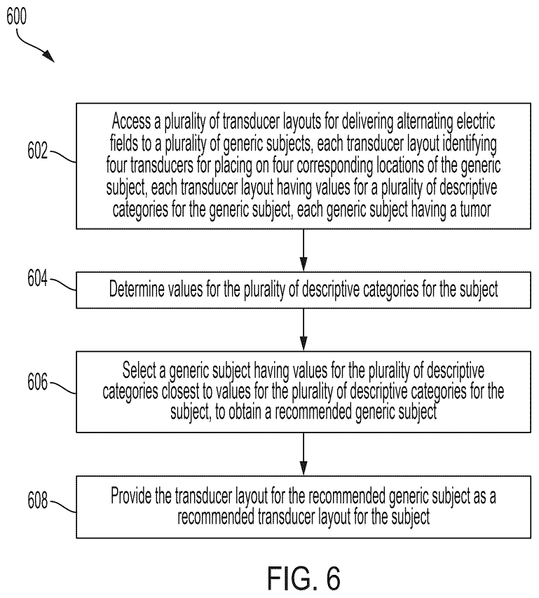

FIG. 6 illustrates a flowchart depicting an example computer-implemented method for selecting transducers for delivering alternating electric fields to a subject. In method 600, the transducers and the transducer layouts are determined via geometrical calculations of the subject. Certain steps of the method 600 may be computer-implemented steps. The computer may be, for example, any device comprising one or more processors and memory accessible by the one or more processors, the memory storing instructions that when executed by the one or more processors cause the computer to perform the relevant steps of the method 600. The method 600 may be implemented by any suitable system or apparatus, such as the apparatus of FIG. 9. While an order of operations is indicated in FIG. 6 for illustrative purposes, the timing and ordering of such operations may vary where appropriate without negating the purpose and advantages of the examples set forth in detail herein.

At step 602, the method 600 may include accessing a plurality of transducer layouts for delivering alternating electric fields to a plurality of generic subjects. The plurality of transducer layouts may be obtained from computer memory (or computer storage) locally or over a network. Each transducer layout may identify four transducers for placing on four corresponding locations of the generic subject. Each transducer layout may have values for a plurality of descriptive categories for the generic subject. The plurality of descriptive categories may include at least a proximity of a ROI to a skin surface of the generic subject. As an example, the plurality of descriptive categories may include a proximity of a ROI to a skin surface and at least one of sex, height, weight, body mass index, age, and location of ROI. In some embodiments, a pair of transducers for at least one of the transducer layouts of the generic subjects may include a small transducer and a large transducer, where the large transducer has an area larger than an area of the small transducer. In some embodiments, a pair of transducers for at least one of the transducer layouts of the generic subjects may correspond to an embodiment for a pair of transducers discussed above in relation to step 108 of FIG. 1.

Referring back to FIG. 6, at step 604, the method 600 may include determining values for each of the plurality of descriptive categories for the subject.

At step 606, the method 600 may include selecting a generic subject having values for the plurality of descriptive categories closest to values for the plurality of descriptive categories for the subject, to obtain a recommended generic subject.

In some embodiments, selecting the generic subject having values for the plurality of descriptive categories closest to values for the plurality of descriptive categories for the subject may include: determining, for each generic subject, a difference between each value of the plurality of descriptive categories of the generic subject and each value of the plurality of descriptive categories of the subject; determining, for each generic subject, a weighted sum of the difference for each value of the plurality of descriptive categories; and selecting the generic subject having a lowest weighted sum as the recommended generic subject.

In some embodiments, the method 600 may further include: selecting a generic subject having values for the plurality of descriptive categories second closest to values for the plurality of descriptive categories for the subject, to obtain a second recommended generic subject; and providing the transducer layout for the second recommended generic subject as a second recommended transducer layout for the subject. In some embodiments, the method 600 may further include: selecting a generic subject having values for the plurality of descriptive categories third closest to values for the plurality of descriptive categories for the subject, to obtain a third recommended generic subject; and providing the transducer layout for the third recommended generic subject as a third recommended transducer layout for the subject.

In some embodiments, the recommended generic subject may be selected based on a first criteria for comparing values of the plurality of descriptive categories between the generic subjects and the subject. In some embodiments, method 600 may further include selecting a generic subject having values for the plurality of descriptive categories closest to values for the plurality of descriptive categories for the subject based on a second criteria, to obtain a second recommended generic subject. In some embodiments, method 600 may further include selecting a generic subject having values for the plurality of descriptive categories closest to values for the plurality of descriptive categories for the subject based on a third criteria, to obtain a third recommended generic subject.

At step 608, the method 600 may include providing the transducer layout for the recommended generic subject as a recommended transducer layout for the subject. In some embodiments, providing the recommended transducer layout for the subject may include displaying an image of the recommended generic subject with the recommended transducer layout depicted on the recommended generic subject. In some embodiments, providing the recommended transducer layout for the subject may include displaying an image of the subject with the recommended transducer layout depicted on the subject. In some embodiments, the method 600 may further include providing the transducer layout for a second recommended generic subject, or additionally providing the transducer layout for a third recommend generic subject.

### Exemplary Apparatuses

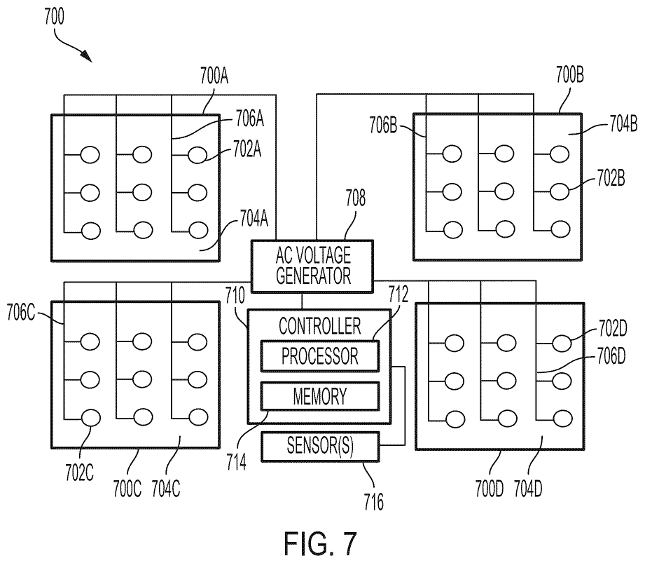

FIG. 7 depicts an example apparatus 700 to apply alternating electric fields (e.g., TTFields) to a subject's body. The system may be used for treating a target region of a subject's body with an alternating electric field (e.g., TTFields). As an example, the target region may be in the subject's brain, and an alternating electric field may be delivered to the subject's body via two pairs of transducer arrays positioned on a head of the subject's body. As another example, the target region may be in the subject's torso, and an alternating electric field may be delivered to the subject's body via two pairs of transducer arrays positioned on at least one of a thorax, an abdomen, or one or both thighs of the subject's body. Other transducer array placements on the subject's body may be possible.

The example apparatus 700 has four transducers (or “transducer arrays”) 700A-D. Each transducer 700A-D may include substantially flat electrode elements 702A-D positioned on a substrate 704A-D and electrically and physically connected (e.g., through conductive wiring 706A-D). The substrates 704A-D may include, for example, cloth, foam, flexible plastic, and/or conductive medical gel. Two transducers (e.g., 700A and 700D) may be a first pair of transducers configured to apply an alternating electric field to a target region of the subject's body. The other two transducers (e.g., 700B and 700C) may be a second pair of transducers configured to similarly apply an alternating electric field to the target region.

The transducers 700A-D may be coupled to an AC voltage generator 708, and the system may further include a controller 710 communicatively coupled to the AC voltage generator 708. The controller 710 may include a computer having one or more processors 712 and memory 714 accessible by the one or more processors. The memory 714 may store instructions that when executed by the one or more processors control the AC voltage generator 708 to induce alternating electric fields between pairs of the transducers 700A-D according to one or more voltage waveforms and/or cause the computer to perform one or more methods disclosed herein. The controller 710 may monitor operations performed by the AC voltage generator 708 (e.g., via the processor(s) 712). One or more sensor(s) 716 may be coupled to the controller 710 for providing measurement values or other information to the controller 710.

The electrode elements 702A-D may be capacitively coupled. In one example, the electrode elements 702A-D may be ceramic electrode elements coupled to each other via conductive wiring 706A-D. When viewed in a direction perpendicular to its face, the ceramic electrode elements may be circular shaped or non-circular shaped. In other embodiments, the array of electrode elements may not be capacitively coupled, and there may be no dielectric material (such as ceramic, or high dielectric polymer layer) associated with the electrode elements.

The structure of the transducers 700A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer may include a polymer film that is adapted to generate an alternating field.

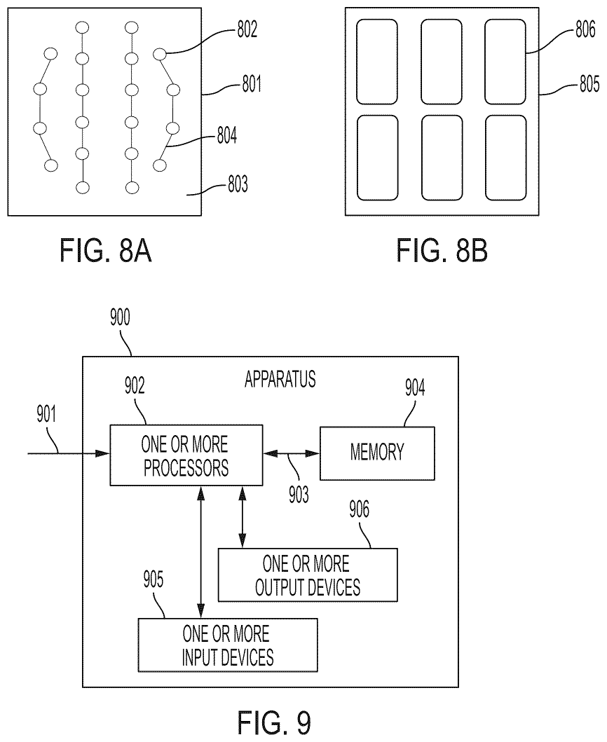

FIG. 8A illustrates a schematic view of an exemplary design of a transducer for applying alternating electric fields. Transducer 801 includes twenty electrode elements 802, which are positioned on substrate 803, and electrode elements 802 are electrically and physically connected to one another through a conductive wiring 804. In some embodiments, electrode elements 802 may include a ceramic disk.

FIG. 8B illustrates a schematic view of an exemplary design of a transducer for applying alternating electric fields. Transducer 805 may include substantially flat electrode elements 806. In some embodiments, electrode elements 806 may be non-ceramic dielectric materials positioned over flat conductors. Examples of non-ceramic dielectric materials positioned over flat conductors may include polymer films disposed over pads on a printed circuit board or over substantially planar pieces of metal. In some embodiments, such polymer films have a high dielectric constant, such as, for example, a dielectric constant greater than 10. In some embodiments, electrode elements 806 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 806 may have a same shape, similar shapes, and/or different shapes.

FIG. 9 depicts an example computer apparatus for use with the embodiments herein. As an example, the apparatus 900 may be a computer to implement certain inventive techniques disclosed herein, such as selecting transducer locations for delivering TTFields to a subject. For example, steps of FIG. 1, FIG. 5, and/or FIG. 6 may be performed by a computer, such as the apparatus 900. As an example, the apparatus 900 may be used as the controller 710 of FIG. 7, or as a separate computer apparatus located remote from the controller 710. The apparatus 900 may include one or more processors 902, memory 904, one or more input devices 905, and one or more output devices 906.

Input to the apparatus 900 may be provided by one or more input devices 905, provided from one or more input devices in communication with the apparatus 900 via link 901 (e.g., a wired link or a wireless link; e.g., with a direct connection or over a network), and/or provided from another computer(s) in communication with the apparatus 900 via link 901.

Output for the apparatus 900 may be provided by one or more output devices 906, provided to one or more output devices in communication with the apparatus 900 via link 901, and/or provided from another computer(s) in communication with the apparatus 900 via link 901. The one or more output devices 906 may include one more displays and one or more speakers. The one or more output devices 906 may provide the status of the operation of the invention, such as transducer location selection, voltages being generated, and other operational information. The output device(s) 906 may provide visualization data according to certain embodiments of the invention.

In some embodiments, one or more input devices 905 and one or more output devices 906 may be combined into one or more unitary input/output devices (e.g., a touch screen).

In some embodiments, based on input from one or more input devices 905 and/or input from outside the apparatus 900 via the link 901, the one or more processors 902 may perform operations as described herein. As an example, user input may be received from the one or more input devices 905. As an example, input may be from another computer in communication with the apparatus 900 via link 901. As an example, input may be from one or more input devices in communication with the apparatus 900 via link 901. As an example, the input may be user input via input device(s) 905 and/or link 901.

In some embodiments, the one or more processors 902 may perform operations as described herein and provide results of the operations as output. As an example, output may be provided to the one or more output devices 906. As an example, output may be provided to another computer in communication with the apparatus 900 via link 901. As an example, output may be provided to one or more output devices in communication with the apparatus 900 via link 901. In some embodiments, based on input from link 901 and/or input device(s) 905, the one or more processors 902 may generate control signals to control the AC voltage generator 708.

The memory 904 may be accessible by the one or more processors 902 (e.g., via link 903) so that the one or more processors 902 may read information from and write information to the memory 904. The memory 904 may store instructions that, when executed by the one or more processors 902, implement one or more embodiments described herein. The memory 904 may be a non-transitory computer readable medium (or a non-transitory processor readable medium) containing a set of instructions thereon for selecting transducer locations for delivering tumor treating fields to a subject, wherein when executed by a processor (such as one or more processors 902), the instructions cause the processor to perform one or more methods discussed herein.

The apparatus 900 may be an apparatus for selecting transducer locations for delivering tumor treating fields to a subject, the apparatus including: one or more processors (such as one or more processors 902); and memory (such as memory 904) accessible by the one or more processors, the memory storing instructions that when executed by the one or more processors, cause the apparatus to perform one or more methods described herein.

The memory 904 may be a non-transitory processor readable medium containing a set of instructions thereon for selecting transducer locations for delivering tumor treating fields to a subject, wherein when executed by a processor (such as processor 902), the instructions cause the processor to perform one or more methods described herein.

### Illustrative Embodiments

The invention includes other illustrative embodiments (“Embodiments”) as follows.

Embodiment 1: A computer-implemented method for selecting transducers for delivering alternating electric fields to a subject, the method comprising: obtaining a three-dimensional model of at least a portion of the subject; determining a location of a region of interest (ROI) in the three-dimensional model; determining first and second potential surfaces of the three-dimensional model-for placement of first and second transducers on the subject based on the ROI; and selecting a first pair of transducers from a plurality of transducers to deliver alternating electric fields to the ROI at selected locations on the three-dimensional model based on the location of the ROI in the three-dimensional model and the potential surfaces of the three-dimensional model, wherein the plurality of transducers comprises a small transducer and a large transducer having an area larger than an area of the small transducer.

Embodiment 2: The method of Embodiment 1, wherein selecting the first pair of transducers from the plurality of transducers comprises: selecting the small transducer as a first transducer of the first pair of transducers; and selecting either the small transducer or the large transducer as a second transducer of the first pair of transducers.

Embodiment 3: The method of Embodiment 1, wherein a first transducer of the first pair of transducers is selected to be the small transducer from the plurality of transducers, wherein a first location of the first transducer is selected to be located on the first surface of the three-dimensional model, wherein a second transducer of the first pair of transducers is selected from the plurality of transducers, wherein a second location of the second transducer is selected to be located on the second potential surface.

Embodiment 4: The method of Embodiment 3, wherein the second transducer of the first pair of transducers is selected from the plurality of transducers based on a proximity of the ROI to the first potential surface of the three-dimensional model.

Embodiment 5: The method of Embodiment 3, wherein the second transducer of the first pair of transducers is selected to be: the large transducer if the ROI is closer to the first potential surface of the three-dimensional model than to midway between the first location and the second location, or the small transducer if the ROI is closer to midway between the first location and the second location on the three-dimensional model.

Embodiment 6: The method of Embodiment 3, wherein a line passing through the ROI is defined between the first location of the first transducer and the second location of the second transducer, wherein the second transducer of the first pair of transducers is selected to be: the small transducer if the ROI is located at about a center of the line, or the large transducer otherwise.

Embodiment 7: The method of Embodiment 3, wherein a line passing through the ROI is defined between the first location of the first transducer and the second location of the second transducer, wherein the second transducer of the first pair of transducers is selected to be: the large transducer if the ROI is located away from a center of the line, or the small transducer otherwise.

Embodiment 7A: The method of Embodiment 3, wherein a line passing through the ROI is defined between the first location of the first transducer and the second location of the second transducer, wherein the second transducer of the first pair of transducers is selected to be: the small transducer if at least about 50% of the ROI is located within about 10% of a center of the line, or the large transducer otherwise.

Embodiment 7B: The method of Embodiment 3, wherein a line passing through the ROI is defined between the first location of the first transducer and the second location of the second transducer, wherein the second transducer of the first pair of transducers is selected to be: the large transducer if at least about 50% the ROI is located more than about 10% from a center of the line, or the small transducer otherwise.

Embodiment 8: The method of Embodiment 1, wherein selecting the first pair of transducers from the plurality of transducers comprises selecting the first pair of transducers from the plurality of transducers based on a size of the ROI in relation to the first potential surface of the three-dimensional model.

Embodiment 9: The method of Embodiment 1, wherein selecting the first pair of transducers from the plurality of transducers comprises: when viewed from a direction approximately perpendicular to the first potential surface of the three-dimensional model, determining an area of the ROI, selecting a transducer from the plurality of transducers having an area closest to the area of the ROI as a first transducer of the first pair of transducers, selecting a first location of the first transducer to be on the first potential surface of the three-dimensional model.

Embodiment 10: The method of Embodiment 11, wherein the plurality of transducers further comprises at least one mid-sized transducer having an area larger than the area of the small transducer and smaller than the area of the large transducer.

Embodiment 11: The method of Embodiment 1, wherein selecting the first pair of transducers from the plurality of transducers comprises: when viewed from a direction approximately perpendicular to the first potential surface of the three-dimensional model, determining a projection of the ROI onto the first potential surface; selecting a transducer from the plurality of transducers having a size closest to a size of the projection of the ROI onto the first potential surface as a first transducer of the first pair of transducers; selecting a first location of the first transducer to be on the first potential surface of the three-dimensional model based on a location of the projection of the ROI onto the first potential surface.

Embodiment 12: The method of Embodiment 11, wherein selecting the first pair of transducers from the plurality of transducers further comprises: when viewed from a direction approximately perpendicular to the first potential surface of the three-dimensional model, determining an area of the projection of the ROI, wherein the transducer selected as the first transducer has an area closest to the area of the projection of the ROI.

Embodiment 13: The method of Embodiment 11, wherein a second transducer of the first pair of transducers is selected to be the large transducer, wherein a second location of the second transducer is selected to be located on the second potential surface of the three-dimensional model, wherein the projection of the ROI onto the first potential surface is based on at least one line intersecting a perimeter of the second transducer and a perimeter of the ROI.

Embodiment 14: The method of Embodiment 1, wherein a first transducer of the first pair of transducers is selected from the plurality of transducers based on a size of the ROI in relation to the first potential surface of the three-dimensional model, wherein a first location of the first transducer is selected to be located on the first potential surface of the three-dimensional model, wherein a second transducer of the first pair of transducers is selected to be the large transducer, wherein a second location of the second transducer is selected to be located on a surface of the three-dimensional model opposite to the first location.

Embodiment 15: The method of Embodiment 1, wherein when viewed from a direction perpendicular to a surface of a transducer, an area of the transducer is an area of the transducer to emanate an electric field when the transducer is adjacent to a surface of three-dimensional model.

Embodiment 16: The method of Embodiment 1, wherein when viewed from a direction perpendicular to a surface of a transducer, an area of the transducer is a summation of areas of all electrodes of the transducer.

Embodiment 16A: The method of Embodiment 1, wherein when viewed from a direction perpendicular to a surface of a transducer, an area of the transducer is an area of a smallest convex shape enclosing all electrodes of the transducer.

Embodiment 16B: The method of Embodiment 1, wherein when viewed from a direction perpendicular to a surface of a transducer, an area of the transducer is an area of a smallest convex shape enclosing electrodes of the transducer to deliver alternating electric fields as part of a pair of transducers.

Embodiment 17: The method of Embodiment 1, wherein the small transducer and the large transducer are each modeled to provide a same amount of current to the subject.

Embodiment 17A: The method of Embodiment 1, wherein the area of the small transducer is about 50% to about 70% of the area of the large transducer.

Embodiment 17B: The method of Embodiment 1, wherein the area of the small transducer is about 150 cm² to about 265 cm², and wherein the area of the large transducer is about 300 cm² to about 525 cm².

Embodiment 17C: The method of Embodiment 1, wherein the plurality of transducers further comprises at least one mid-sized transducer having an area larger than the area of the small transducer and smaller than the area of the large transducer.

Embodiment 18: The method of Embodiment 1, wherein the potential surfaces of the three-dimensional model are determined based on at least one of a centroid of the ROI, an exterior surface of the ROI, or a concave shape enclosing the ROI.

Embodiment 18A: The method of Embodiment 1, wherein determining the first potential surface of the three-dimensional model comprises: determining a centroid of the ROI; determining a point on the surface of the three-dimensional model of the subject closest to the centroid of the ROI, wherein the first potential surface includes the point.

Embodiment 18B: The method of Embodiment 1, wherein determining the first potential surface of the three-dimensional model comprises: determining an exterior surface of the ROI; determining a point on the surface of the three-dimensional model of the subject closest to the exterior surface of the ROI, wherein the first potential surface includes the point.

Embodiment 18C: The method of Embodiment 1, wherein determining the first potential surface of the three-dimensional model comprises: determining a concave shape enclosing the ROI; determining a point on the surface of the three-dimensional model of the subject closest to the concave shape of the ROI, wherein the first potential surface includes the point.

Embodiment 18D: The method of Embodiment 1, further comprising: selecting a second pair of transducers from the plurality of transducers to deliver alternating electric fields to the ROI at selected locations on the three-dimensional model.

Embodiment 18E: The method of Embodiment 1, further comprising: outputting the first pair of transducers and the locations for first pair of transducers.

Embodiment 19: A non-transitory processor readable medium containing a set of instructions thereon for selecting transducers for delivering alternating electric fields to a subject, wherein when executed by a processor, the instructions cause the processor to perform a method comprising: obtaining a three-dimensional model of at least a portion of the subject; determining a location of a ROI in the three-dimensional model; determining first and second potential surfaces of the three-dimensional model for placement of first and second transducers on the subject based on the ROI; and selecting a first pair of transducers from a plurality of transducers to deliver alternating electric fields to the ROI at selected locations on the three-dimensional model based on the location of the ROI in the three-dimensional model and the potential surfaces of the three-dimensional model, wherein the plurality of transducers comprises a small transducer and a large transducer having an area larger than an area of the small transducer.

Embodiment 20: An apparatus for selecting transducers for delivering alternating electric fields to a subject, the apparatus comprising: one or more processors; and memory accessible by the one or more processors, the memory storing instructions that when executed by the one or more processors, cause the apparatus to perform a method comprising: obtaining a three-dimensional model of at least a portion of the subject; determining a location of a ROI in the three-dimensional model; determining first and second potential surfaces of the three-dimensional model for placement of first and second transducers on the subject based on the ROI; and selecting a first pair of transducers from a plurality of transducers to deliver alternating electric fields to the ROI at selected locations on the three-dimensional model based on the location of the ROI in the three-dimensional model and the potential surfaces of the three-dimensional model, wherein the plurality of transducers comprises a small transducer and a large transducer having an area larger than an area of the small transducer.

Embodiment 21: A computer-implemented method for selecting transducers for delivering alternating electric fields to a subject, the method comprising: storing a plurality of transducer layouts for delivering alternating electric fields to a plurality of generic subjects, each transducer layout identifying four transducers for placing on four corresponding locations of the generic subject, each transducer layout having values for a plurality of descriptive categories for the generic subject, each generic subject having a ROI; determining values for the plurality of descriptive categories for the subject; selecting a generic subject having values for the plurality of descriptive categories closest to values for the plurality of descriptive categories for the subject, to obtain a recommended generic subject; and providing the transducer layout for the recommended generic subject as a recommended transducer layout for the subject, wherein the plurality of descriptive categories comprises a proximity of a ROI to a skin surface.

Embodiment 22: The method of Embodiment 21, wherein each generic subject differs from every other generic subject by a value for at least one descriptive category.

Embodiment 23: The method of Embodiment 21, wherein the plurality of descriptive categories further comprises at least one of sex of subject, height of subject, weight of subject, body mass index of subject, age of subject, or location of ROI in subject.

Embodiment 24: The method of Embodiment 21, wherein the transducers of the transducer layouts of the generic subjects comprises a small transducer and a large transducer having an area larger than an area of the small transducer.

Embodiment 25: The method of Embodiment 21, wherein selecting the generic subject having values for the plurality of descriptive categories closest to values for the plurality of descriptive categories for the subject, comprises: determining, for each generic subject, a difference between each value of the plurality of descriptive categories of the generic subject and each value of the plurality of descriptive categories of the subject; determining, for each generic subject, a weighted sum of the difference for each value of the plurality of descriptive categories; selecting the generic subject having a lowest weighted sum as the recommended generic subject.

Embodiment 26: The method of Embodiment 21, further comprising: selecting a generic subject having values for the plurality of descriptive categories second closest to values for the plurality of descriptive categories for the subject, to obtain a second recommended generic subject; and providing the transducer layout for the second recommended generic subject as a second recommended transducer layout for the subject.

Embodiment 27: The method of Embodiment 26, further comprising: electing a generic subject having values for the plurality of descriptive categories third closest to values for the plurality of descriptive categories for the subject, to obtain a third recommended generic subject; and providing the transducer layout for the third recommended generic subject as a third recommended transducer layout for the subject.

Embodiment 28: The method of Embodiment 21, wherein the recommended generic subject is selected based on a first criteria for comparing values of the plurality of descriptive categories between the generic subjects and the subject, the method further comprising: selecting a generic subject having values for the plurality of descriptive categories closest to values for the plurality of descriptive categories for the subject based on a second criteria, to obtain a second recommended generic subject; and providing the transducer layout for the second recommended generic subject as a second recommended transducer layout for the subject.

Embodiment 29: The method of Embodiment 21, wherein providing the recommended transducer layout for the subject, comprises: providing for display an image of the recommended generic subject with the recommended transducer layout depicted on the recommended generic subject.

Embodiment 30: The method of Embodiment 21, wherein providing the recommended transducer layout for the subject, comprises: providing for display an image of the subject with the recommended transducer layout depicted on the subject.

Embodiment 31: The method of Embodiment 30, wherein the image of the subject is based on at least one of a computer tomography (CT) medical image of the subject, a magnetic resonance imaging (MRI) medical image of the subject, or a positron emission tomography (PET) medical image of the subject.

Embodiment 32: A non-transitory processor readable medium containing a set of instructions thereon for selecting transducers for delivering alternating electric fields to a subject, wherein when executed by a processor, the instructions cause the processor to perform a method comprising: accessing a plurality of transducer layouts for delivering alternating electric fields to a plurality of generic subjects, each transducer layout identifying four transducers for placing on four corresponding locations of the generic subject, each transducer layout having values for a plurality of descriptive categories for the generic subject, each generic subject having a ROI; determining values for the plurality of descriptive categories for the subject; selecting a generic subject having values for the plurality of descriptive categories closest to values for the plurality of descriptive categories for the subject, to obtain a recommended generic subject; and providing the transducer layout for the recommended generic subject as a recommended transducer layout for the subject, wherein the plurality of descriptive categories comprises a proximity of a ROI to a skin surface.

Embodiment 33: A non-transitory processor readable medium containing a set of instructions thereon for selecting transducers for delivering alternating electric fields to a subject, wherein when executed by a processor, the instructions cause the processor to perform a method comprising: accessing a plurality of transducer layouts for delivering alternating electric fields to a plurality of generic subjects, each transducer layout identifying four transducers for placing on four corresponding locations of the generic subject, each transducer layout having values for a plurality of descriptive categories for the generic subject, each generic subject having a ROI; determining values for the plurality of descriptive categories for the subject; selecting a generic subject having values for the plurality of descriptive categories closest to values for the plurality of descriptive categories for the subject, to obtain a recommended generic subject; and providing the transducer layout for the recommended generic subject as a recommended transducer layout for the subject, wherein the plurality of descriptive categories comprises a proximity of a ROI to a skin surface.

Embodiment 34: A method, machine, manufacture, and/or system substantially as shown and described.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A computer-implemented method for selecting transducers for delivering alternating electric fields to a subject, the method comprising:
obtaining a three-dimensional model of at least a portion of the subject;
determining a location of a region of interest (ROI) in the three-dimensional model;
determining first and second potential surfaces of the three-dimensional model for placement of first and second transducers on the subject based on the ROI; and
selecting a first pair of transducers from a plurality of transducers to deliver alternating electric fields to the ROI at selected locations on the three-dimensional model based on the location of the ROI in the three-dimensional model and the potential surfaces of the three-dimensional model,
wherein the plurality of transducers comprises a small transducer and a large transducer having an area larger than an area of the small transducer.

2. The computer-implemented method of claim 1, wherein selecting the first pair of transducers from the plurality of transducers comprises:
selecting the small transducer as a first transducer of the first pair of transducers; and
selecting either the small transducer or the large transducer as a second transducer of the first pair of transducers.

3. The computer-implemented method of claim 1,
wherein a first transducer of the first pair of transducers is selected to be the small transducer from the plurality of transducers,
wherein a first location of the first transducer is selected to be located on the first potential surface of the three-dimensional model,
wherein a second transducer of the first pair of transducers is selected from the plurality of transducers,
wherein a second location of the second transducer is selected to be located on the second potential surface.

4. The computer-implemented method of claim 3, wherein the second transducer of the first pair of transducers is selected from the plurality of transducers based on a proximity of the ROI to the first potential surface of the three-dimensional model.

5. The computer-implemented method of claim 1, wherein selecting the first pair of transducers from the plurality of transducers comprises selecting the first pair of transducers from the plurality of transducers based on a size of the ROI in relation to the first potential surface of the three-dimensional model.

6. The computer-implemented method of claim 1, wherein selecting the first pair of transducers from the plurality of transducers comprises:
when viewed from a direction approximately perpendicular to the first potential surface of the three-dimensional model, determining an area of the ROI,
selecting a transducer from the plurality of transducers having an area closest to the area of the ROI as a first transducer of the first pair of transducers,
selecting a first location of the first transducer to be on the first potential surface of the three-dimensional model.

7. The computer-implemented method of claim 1, wherein selecting the first pair of transducers from the plurality of transducers comprises:
when viewed from a direction approximately perpendicular to the first potential surface of the three-dimensional model, determining a projection of the ROI onto the first potential surface;
selecting a transducer from the plurality of transducers having a size closest to a size of the projection of the ROI onto the first potential surface as a first transducer of the first pair of transducers;
selecting a first location of the first transducer to be on the first potential surface of the three-dimensional model based on a location of the projection of the ROI onto the first potential surface.

8. The computer-implemented method of claim 1, wherein the small transducer and the large transducer are each modeled to provide a same amount of current to the subject.

9. A non-transitory processor readable medium containing a set of instructions thereon for selecting transducers for delivering alternating electric fields to a subject, wherein when executed by a processor, the instructions cause the processor to perform the method of claim 1.

10. An apparatus for selecting transducers for delivering alternating electric fields to a subject, the apparatus comprising: one or more processors; and memory accessible by the one or more processors, the memory storing instructions that when executed by the one or more processors, cause the apparatus to perform a method comprising:
obtaining a three-dimensional model of at least a portion of the subject;
determining a location of a region of interest (ROI) in the three-dimensional model;
determining first and second potential surfaces of the three-dimensional model for placement of first and second transducers on the subject based on the ROI; and
selecting a first pair of transducers from a plurality of transducers to deliver alternating electric fields to the ROI at selected locations on the three-dimensional model based on the location of the ROI in the three-dimensional model and the potential surfaces of the three-dimensional model,
wherein the plurality of transducers comprises a small transducer and a large transducer having an area larger than an area of the small transducer.

11. A computer-implemented method for selecting transducers for delivering alternating electric fields to a subject, the method comprising:
accessing a plurality of transducer layouts for delivering alternating electric fields to a plurality of generic subjects, each transducer layout identifying four transducers for placing on four corresponding locations of the generic subject, each transducer layout having values for a plurality of descriptive categories for the generic subject, each generic subject having a region of interest (ROI);
determining values for the plurality of descriptive categories for the subject;
selecting a generic subject having values for the plurality of descriptive categories closest to values for the plurality of descriptive categories for the subject, to obtain a recommended generic subject; and
providing the transducer layout for the recommended generic subject as a recommended transducer layout for the subject,
wherein the plurality of descriptive categories comprises a proximity of a ROI to a skin surface.

12. The computer-implemented method of claim 11, wherein each generic subject differs from every other generic subject by a value for at least one descriptive category.

13. The computer-implemented method of claim 11, wherein the plurality of descriptive categories further comprises at least one of sex of subject, height of subject, weight of subject, body mass index of subject, age of subject, or location of ROI in subject.

14. The computer-implemented method of claim 11, wherein the transducers of the transducer layouts of the generic subjects comprises a small transducer and a large transducer having an area larger than an area of the small transducer.

15. The computer-implemented method of claim 11, wherein selecting the generic subject having values for the plurality of descriptive categories closest to values for the plurality of descriptive categories for the subject, comprises:
determining, for each generic subject, a difference between each value of the plurality of descriptive categories of the generic subject and each value of the plurality of descriptive categories of the subject;
determining, for each generic subject, a weighted sum of the difference for each value of the plurality of descriptive categories;
selecting the generic subject having a lowest weighted sum as the recommended generic subject.

16. The computer-implemented method of claim 11, wherein the recommended generic subject is selected based on a first criteria for comparing values of the plurality of descriptive categories between the generic subjects and the subject, the method further comprising:
selecting a generic subject having values for the plurality of descriptive categories closest to values for the plurality of descriptive categories for the subject based on a second criteria, to obtain a second recommended generic subject; and
providing the transducer layout for the second recommended generic subject as a second recommended transducer layout for the subject.

17. The computer-implemented method of claim 11, wherein providing the recommended transducer layout for the subject, comprises:
providing for display an image of the subject with the recommended transducer layout depicted on the subject.

18. The computer-implemented method of claim 17, wherein the image of the subject is based on at least one of a computer tomography (CT) medical image of the subject, a magnetic resonance imaging (MRI) medical image of the subject, or a positron emission tomography (PET) medical image of the subject.

19. A non-transitory processor readable medium containing a set of instructions thereon for selecting transducers for delivering alternating electric fields to a subject, wherein when executed by a processor, the instructions cause the processor to perform the method of claim 11.

20. An apparatus for selecting transducers for delivering alternating electric fields to a subject, the apparatus comprising: one or more processors; and memory accessible by the one or more processors, the memory storing instructions that when executed by the one or more processors, cause the apparatus to perform the method of claim 11.
